(19) United States
(12) Patent Application Publication
(10) Pub. No.: US 2015/0272433 A1
(43) Pub. Date: Oct. 1, 2015

(54) OPTICAL MEASURING DEVICE AND METHOD

(71) Applicant: CRYSTALVUE MEDICAL CORPORATION, Gueishan Township (TW)

(72) Inventors: WILLIAM WANG, Taoyuan City (TW); CHUNG-PING CHUANG, Gueishan Township (TW); MENG-SHIN YEN, Taipei City (TW); CHUNG-CHENG CHOU, Luzhu Township (TW)

(21) Appl. No.: 14/559,918

(22) Filed: Dec. 3, 2014

(30) Foreign Application Priority Data
Apr. 1, 2014 (TW) 103112170

(51) Int. Cl. A61B 3/00; A61B 3/10; A61B 3/14; G02B 26/08

(52) U.S. Cl. CPC A61B 3/0075; G02B 26/0816; A61B 3/10; A61B 3/14; A61B 3/0025; G02B 3/0081

(57) ABSTRACT

An optical measuring device includes a light source module, a light coupling module, a reference mirror module and a processing unit. The light source module can provide a light. The light of the light source module is transmitted to the reference mirror module and an under-test object through the light coupling module. The light is reflected by the reference mirror module and the under-test object to form a first light and a second light, respectively. The first and second lights are then transmitted to the processing unit through the light coupling module. The processing unit generates an adjusting signal according to the first and second lights. The processing unit transmits the adjusting signal to the reference mirror module. The reference mirror module adjusts the reference mirror module according to the adjusting signal.

### CROSS REFERENCE TO RELATED APPLICATIONS

This Non-provisional application claims priority under 35 U.S.C. §119(a) on Patent Application No(s). 103112170 filed in Taiwan, Republic of China on Apr. 1, 2014, the entire contents of which are hereby incorporated by reference.

### BACKGROUND OF THE INVENTION

1. Field of Invention

The invention relates to an optical measuring device and method.

2. Related Art

Due to the advantages of noninvasive detection and rapid reaction, the optical measuring technique is often applied to the noncontact measurement, such as the physiological detection, especially for the ocular detection undergoing high light permeability and easy injury.

FIG. 1 is a schematic diagram of a conventional optical measuring device. As shown in FIG. 1, the optical measuring device 1 at least includes a light source module 10, a reference mirror module 12, a light coupling module 14 and a processing unit 16.

The light provided by the light source module 10 is transmitted to the reference mirror module 12 and the under-test object O through the light coupling module 14 and then reflected by the reference mirror module 12 and the under-test object O to form the reference light R1 and the detection light D1 which are then transmitted to the processing unit 16. Subsequently, the surface curvature of the under-test object O can be acquired according to the interference of the reference light R1 and the detection light D1. Besides, the user can move the reference mirror module 12 to make the reference light R1 and the detection light D1 interfere with each other.

However, the shortcoming of such kind of optical measuring device is that the surface curvature of the under-test object needs to be identified as a concave, convex or flat surface before the measuring starts. Besides, in order to obtain more accurate measurement result, the curvature of the selected reference mirror module 12 needs to approach that of the under-test object (the erroneously selected mirror will increase the error of the measurement result). In other words, this kind of optical measuring device 1 can't be applied to the case such as the irregular surface, the multi-layer object or the high accuracy requirement.

Therefore, it is an important subject to provide an optical measuring device which can increase the measurement accuracy and can be applied to the irregular surface, continuous detection and object of multi-layer structure.

### SUMMARY OF THE INVENTION

In view of the foregoing subject, an objective of the invention is to provide an optical measuring device including a light source module, a light coupling module, a reference mirror module and a processing unit.

The light source module can provide a light. The light of the light source module is transmitted to the reference mirror module and an under-test object through the light coupling module. The light is reflected by the reference mirror module and the under-test object to form a first light and a second light, respectively. The first and second lights are then transmitted to the processing unit through the light coupling module. The processing unit generates an adjusting signal according to the first and second lights. The processing unit transmits the adjusting signal to the reference mirror module. The reference mirror module adjusts the reference mirror module according to the adjusting signal.

In one embodiment, the reference mirror module includes an actuator and a reflector, and the actuator adjusts a curvature of the reflector according to the adjusting signal.

In one embodiment, the reference mirror module includes a light path adjusting unit and a plurality of reference mirrors, and the light path adjusting unit matches the second light with one of the reference mirrors according to the adjusting signal.

In one embodiment, each of the reference mirrors has different curvature.

In one embodiment, the reference mirror module includes an electro-wetting curvature lens or a dielectrophoretic curvature lens.

In one embodiment, the under-test object is a spherical body having a plurality of curved surfaces.

In one embodiment, the spherical body is an eyeball.

An optical measuring method is further provided by this invention and comprises the steps of: providing a light to a reference mirror module and another light to an under-test object; the light reflected by the reference mirror module to form a first light and the other light reflected by the under-test object to form a second light.

The steps further comprise: the first light and the second light interfering with each other; and determining whether the interference conforms to a predetermined interference range, if not, generating an adjusting signal according to the interference and adjusting the reference mirror module by the reference mirror module according to the adjusting signal.

In one embodiment, the reference mirror module adjusts a curvature of the reference mirror module according to the adjusting signal.

In one embodiment, the reference mirror module includes an actuator and a reflector and the steps further comprise: adjusting a curvature of the reflector by the actuator according to the adjusting signal.

In one embodiment, the reference mirror module includes a light path adjusting unit and a plurality of reference mirrors and each of the reference mirrors has different curvature, and the steps further comprise: matching the second light with one of the reference mirrors by the light path adjusting unit according to the adjusting signal.

In one embodiment, the steps further comprise: forming an image of an under-test surface of the under-test object according to the interference.

In one embodiment, the under-test object has a plurality of curved surfaces, and the steps further comprise: repeating the measuring manner to measure the curved surfaces.

In one embodiment, the steps further comprise: superposing the curved surfaces to form a stereoscopic image.

As mentioned above, in this invention, the optical property of the reference mirror module is adjusted by the interference result between the first and second lights, and therefore the measurement accuracy can be enhanced and the shortcoming that the reference mirror module needs to be, in advance, configured with a proper curvature range in response to different curved surfaces or multi-layer object can be overcome.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a schematic diagram of a conventional optical measuring device;

FIG. 2A is a schematic diagram of the optical measuring device of the first embodiment of the invention;

FIGS. 2B and 2C are schematic enlarged diagrams of the reference mirror module of FIG. 2A;

FIG. 3 is a schematic flowchart of the operation of the optical measuring device of the invention;

FIG. 4A is a schematic diagram of the reference mirror module of the optical measuring device of the second embodiment of the invention;

FIGS. 4B and 4C are schematic sectional diagrams taken along the lines AA and BB in FIG. 4A;

FIG. 4D is a schematic diagram of the operation of the reference mirror module in FIG. 4A;

FIG. 5 is a schematic diagram of the reference mirror module of the optical measuring device of the third embodiment of the invention;

FIG. 6 is a schematic diagram of the reference mirror module of the optical measuring device of the fourth embodiment of the invention;

FIGS. 7A to 7C are schematic diagrams of the reference mirror module of the optical measuring device of the fifth embodiment of the invention;

FIGS. 8A to 8C are schematic diagrams of the reference mirror module of the optical measuring device of the sixth embodiment of the invention; and

FIGS. 9A to 9C are schematic diagrams of the reference mirror module of the optical measuring device of the seventh embodiment of the invention.

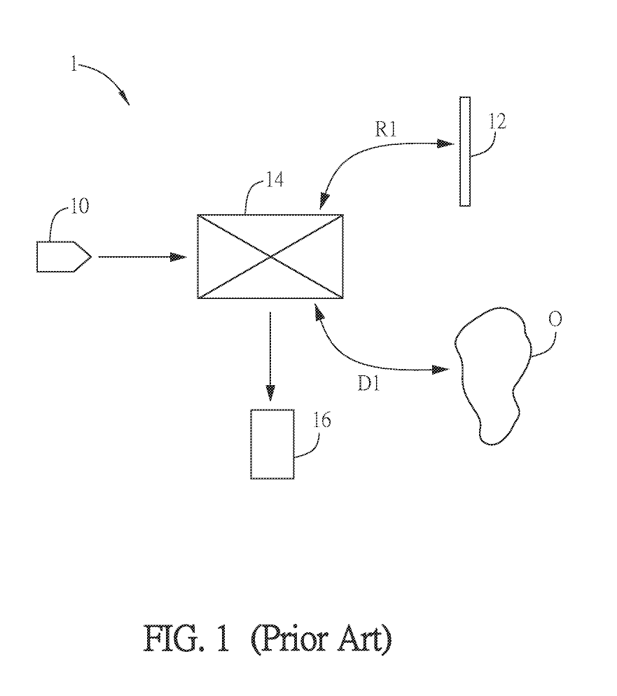

### DETAILED DESCRIPTION OF THE INVENTION

The present invention will be apparent from the following detailed description, which proceeds with reference to the accompanying drawings, wherein the same references relate to the same elements.

To be noted, in the following embodiments and figures, the elements and steps not directly related to this invention are omitted and not shown, and besides, the dimensional relationship between the elements in the figures is just for the easier understanding and not meant to be construed in a limiting sense.

As below, an optical measuring device and method of an embodiment of the invention are illustrated.

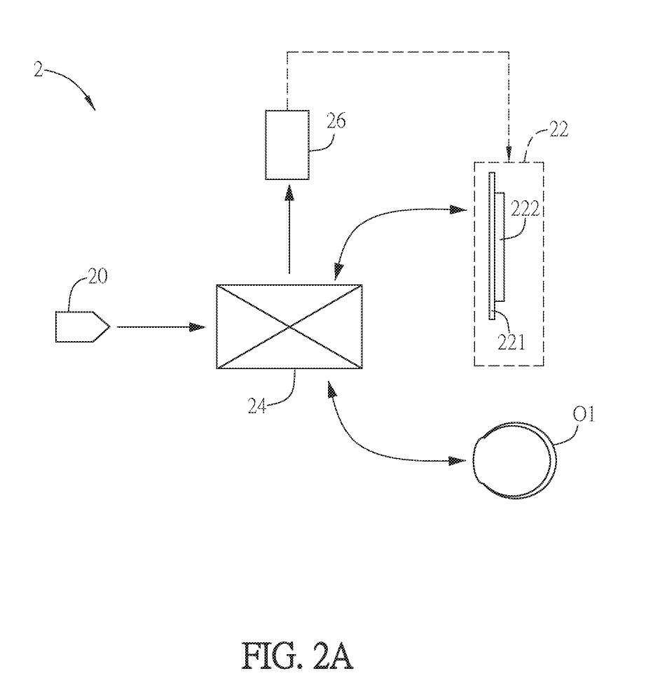

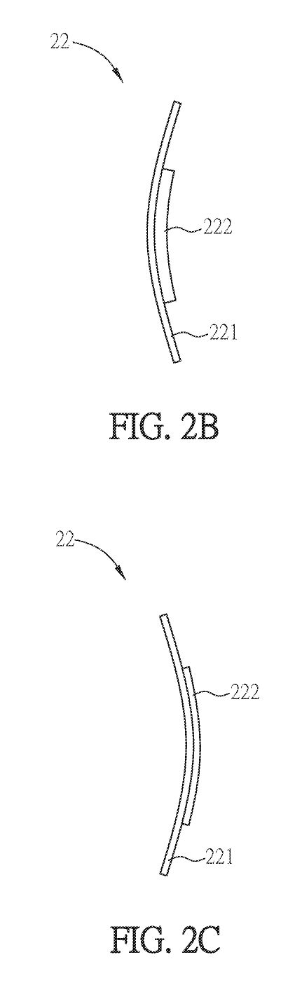

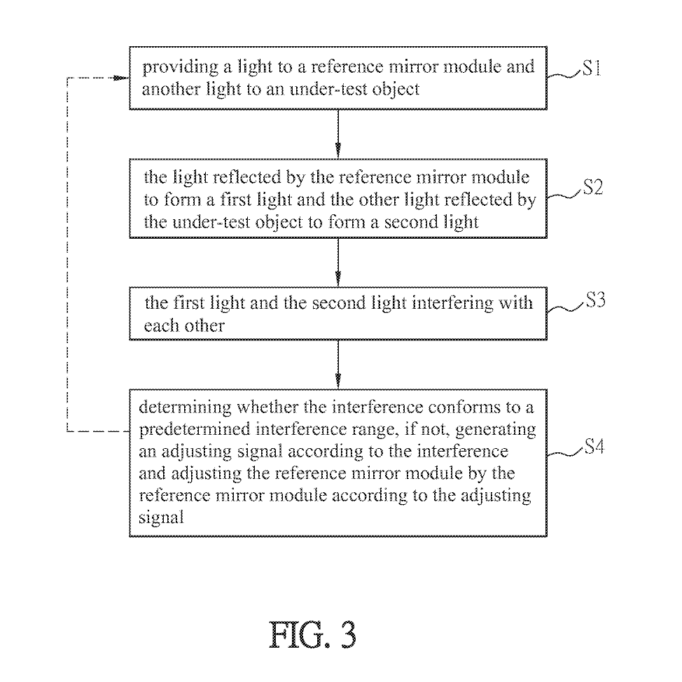

Refer to FIGS. 2A, 2B, 2C and 3. FIG. 2A is a schematic diagram of the optical measuring device of the first embodiment of the invention, FIGS. 2B and 2C are schematic enlarged diagrams of the reference mirror module of FIG. 2A, and FIG. 3 is a schematic flowchart of the operation of the optical measuring device of the invention.

The optical measuring device 2 of this embodiment at least can include a light source module 20, a light coupling module 24, a reference mirror module 22 and a processing unit 26.

The light source module 20 can provide a light. When applied to the human corneal or retinal detection, the light source module 20 can be a wideband laser source for making the testee feel more comfortable. For example, the wavelength thereof can be adjusted to about 840 nm when applied to the retinal detection, to about 1060 nm or 1310 nm when applied to the corneal detection and to about 1310 nm when applied to the skin detection. The bandwidth of the light source module is about 20 nm-40 nm. In other words, the actual wavelength and bandwidth can be adjusted according to the type of the applied target.

The light coupling module 24 can transmit the light of the light source module 20 to the reference mirror module 22 and the under-test object O1. The light coupling module 24 of this embodiment can be a light splitter for example, but this invention is not limited thereto. For example, the 50% light of the light source module 20 can be reflected to enter the reference mirror module 22 and the rest 50% light can penetrate to enter the under-test object O1 so that the light coupling effect can be achieved. Furthermore, the under-test object O1 of this embodiment is a spherical body such as an eyeball, but this invention is not limited thereto.

The reference mirror module 22 of this embodiment can include an actuator (such as a micro-actuator) 221 and a reflector 222 that is made by flexible material and attached to the actuator 221. The actuator 221 can adjust the curvature of the reflector 222 as a convex surface (FIG. 2B), concave surface (FIG. 2C) or flat surface (FIG. 2A) for example according to the signal provided by the processing unit 26. In other words, the deformation level of the reflector 222 can be adjusted by the actuator 221 according to different adjusting signals, so that the curvature of the reflector 222 is changed for obtaining a better measurement result. The method of generating the adjusting signal will be illustrated as below.

Moreover, the reference mirror module 22 of this embodiment can move in a reciprocating manner (by a transmission platform for example) to result in a better interference effect.

First, the light source module 20 can provide a light to the reference mirror module 22 and another light to the under-test object (step S1). Herein for example, the light of the light source module 20 can be transmitted to the reference mirror module 22 and the under-test object O1 through the light coupling module 24.

Then, the light is reflected by the reference mirror module 22 to form a first light and the other light is reflected by the under-test object O1 to form a second light (step S2). In other words, the light transmitted to the reference mirror module 22 and the surface of the under-test object O1 are both reflected. Furthermore, the reflected first and second lights are transmitted to the processing unit 26 through the light coupling module 24.

The first light and the second light interfere with each other (step S3). Meanwhile, the processing unit 26 can record a relative optical path length (optical path difference, OPD) between the first and second lights for serving as the subsequent judgment basis. In detail, the processing unit 26 can include a storage unit which can store the interference results corresponding to different optical path lengths. In other words, it can be achieved by a look-up table manner whether the optical path length conforms to a predetermined interference range or not. If not, an adjusting signal can be generated according to the interference, and the reference mirror module 22 can adjust the reference mirror module 22 according to the adjusting signal (step S4).

In this embodiment, the processing unit 26 will look up the corresponding correction according to the interference result of the first and second lights (a database inside the processing unit 26 can provide the adjustment manner, such as increasing or decreasing the curvature, corresponding to the interference result) for providing an adjusting signal. Then, the processing unit 26 will transmit the adjusting signal to the reference mirror module 22, and the reference mirror module 22 will adjust the reference mirror module 22 according to the adjusting signal (that is, the actuator 221 will deform the reflector 222 to change the curvature of the reflector).

For example, the first measuring can be implemented when the curvature of the reference mirror module 22 is predetermined as zero (i.e. a flat surface), and then the following measuring is implemented when the curvature of the reference mirror module 22 is adjusted as a convex and concave surface. The processing unit 26 will compare these measurement results and adjust the reference mirror module 22 so that its curvature can be closer to the curvature of the under-test object so as to obtain more accurate measurement result.

Otherwise, in the situation of roughly knowing the curvature range of the surface of the under-test object (maybe the age and corneal condition of the testee are known for example), the processing unit 26 can, before the measuring, look up the average value of the corneal curvature corresponding to the age in the database and set the average value as the curvature of the reference mirror module 22 for the first measuring and as the subsequent adjustment basis. Or, the reference mirror module can be first adjusted as a convex surface (cornea is a convex surface) and then the curvature of the convex surface is reduced gradually while the interference of the first and second lights is measured continuously. If the interference situation is worse, the processing unit will transmit the adjusting signal to stop the decrement of the curvature of the convex surface and increase the curvature of the convex surface until the optimum situation occurs.

In other words, the above adjustment will result in the more accurate interference of the first and second lights and the surface curvature of the under-test object O1 can be measured thereby. To be noted, the above adjustment or correction manner can adjust the surface curvature (or range) of the reference mirror module 22 to approach that of the under-test object O1. Furthermore, the measuring and adjustment can be repeatedly implemented according to the requirement until the desired accuracy is achieved.

Moreover, the optical measuring device 2 of this embodiment can further include an image analysis unit (not shown), which can be used to analyze and construct the surface or stereoscopic image of the under-test object. Physically, the image analysis unit can be a charge-coupled device (CCD) camera or a complementary metal-oxide-semiconductor (CMOS) camera.

Accordingly, the steps of this embodiment further include forming the image of the under-test surface of the under-test object according to the interference. Through the calculation, the figure (planar image) of the surface of the under-test object O1 can be plotted and constructed by using the relative optical path lengths measured at many points and regions of the under-test object O1. The plot scheme can be performed by the calculation of interferometric surface profiling, but this invention is not limited thereto.

Moreover, if the under-test object has an irregular curved surface, the surface of the under-test object can be divided into a plurality of regions (such as annular regions or checkerboard-like regions). Then, the above-mentioned adjustment and measuring can be implemented to each of the regions to form the surface image of the under-test object.

Or, if the under-test object has a plurality of curved surfaces or layers, each of the curved surfaces can be adjusted and measured and then the curved surfaces of different depths can be superposed to form the stereoscopic image.

Or, if this embodiment is applied to the eyeball measuring, the structure such as cornea, corneal thickness and retina can be also measured and the stereoscopic image of the eyeball can be directly formed, whereas different reference mirrors are required for the optical measuring device of the conventional art to measure the cornea and retina (that means two or more measuring instruments are necessary for the conventional art). In other words, the optical measuring device of this embodiment at least can include the following advantages: saving the cost (the same optical measuring device can fit different curvatures), saving the time (saving the time of replacing the instruments), and achieving more accurate measurement result (the reference mirror module is adjustable to more approach the surface curvature of the under-test object).

In other words, through the interference result of the first and second lights, it can be found whether the curvature of the reference mirror module fits that of the under-test object. Besides, the curvature of the reference mirror module can be adjusted by the adjusting signal so as to match the under-test surface (or local surface) of the under-test object, and therefore the measurement accuracy can be enhanced. Furthermore, this embodiment also can overcome the shortcoming that the reference mirror module needs to be, in advance, configured with a proper curvature range in response to different curved surfaces or multi-layer object.

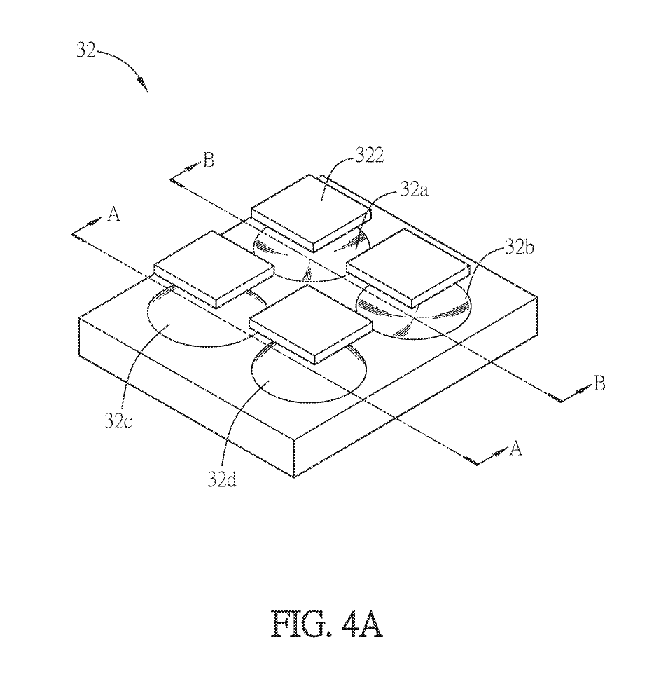

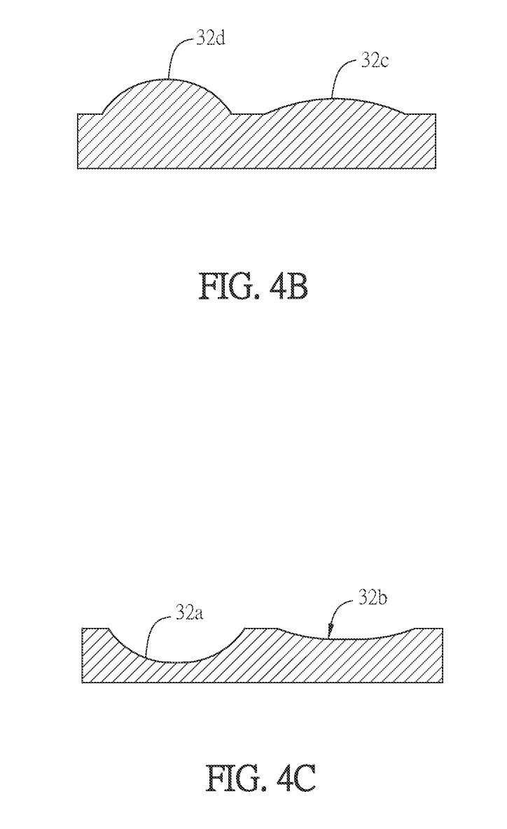

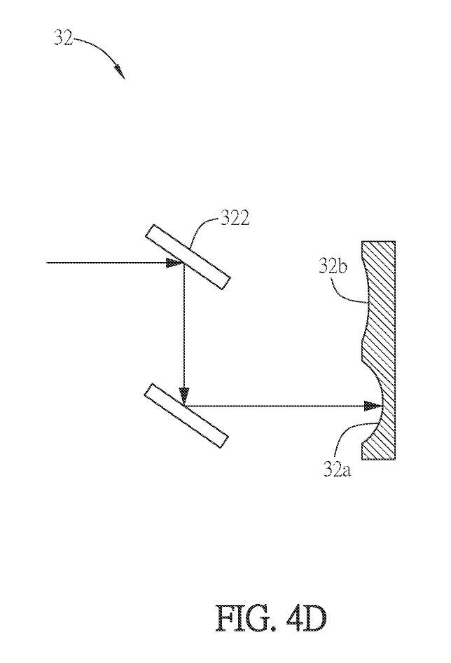

Then, refer to FIGS. 4A to 4D. FIG. 4A is a schematic diagram of the reference mirror module of the optical measuring device of the second embodiment of the invention, FIGS. 4B and 4C are schematic sectional diagrams taken along the lines AA and BB in FIG. 4A, and FIG. 4D is a schematic diagram of the operation of the reference mirror module in FIG. 4A.

In this embodiment, the reference mirror module 32 can include a light path adjusting unit 322 and a plurality of reference mirrors 32a, 32b, 32c, 32d. The light path adjusting unit 322 can match the second light with a reference mirror according to the inputted adjusting signal. That is, the light path adjusting unit 322 can change the incident angle of the light according to the adjusting signal so that the light can enter different reference mirrors. Moreover, the reference mirrors 32a, 32b, 32c, 32d can have different curvatures. Although the reference mirrors 32a, 32b, 32c, 32d of this embodiment are arranged in a matrix of 2×2, this invention is not limited thereto. Besides, the quantity of the reference mirrors is just for the illustration and can be changed according to the requirements.

The light path adjusting unit 322 of this embodiment can include a plurality of reflectors. A driving device (not shown) can be disposed so that the rotation of the reflector can be adjusted according to the adjusting signal and the light can be thus emitted to the reference mirrors with different curvatures (FIG. 4D). Therefore, the measurement accuracy can be enhanced. For example, if the curvature of the surface of the under-test object is measured as closer to the reference mirror 32b, the processing unit will transmit the adjusting signal to rotate the reflector that corresponds to the reference mirror 32b, so that the light is emitted to the reference mirror 32b. In other words, the reference mirror 32b will serve as the reference mirror surface of the under-test object.

Since the relations of other components are similar to the above embodiment, the related descriptions are omitted here for conciseness.

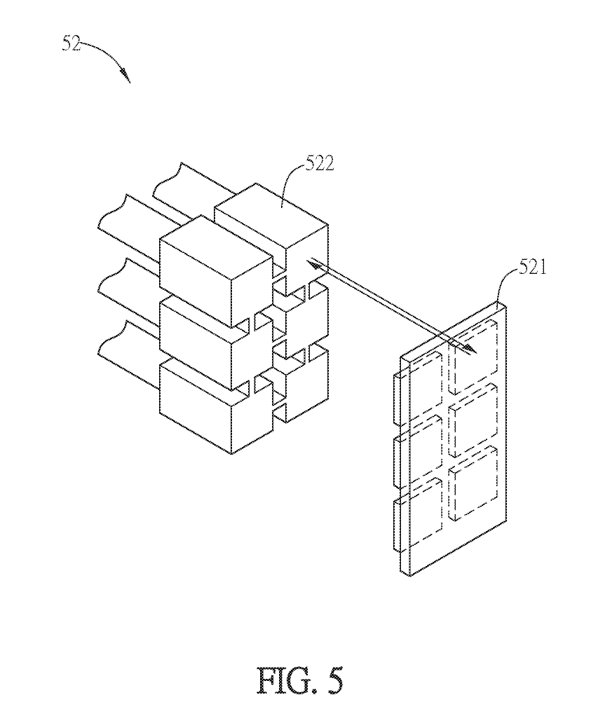

Refer to FIG. 5, which is a schematic diagram of the reference mirror module of the optical measuring device of the third embodiment of the invention.

The main difference from the above-mentioned reference mirror module 32 in FIG. 4A is that the reference mirror module 52 of this embodiment is composed of an optical fiber array 522 and reference mirrors 521, each of the optical fibers corresponds to a reference mirror and the reference mirrors also can have different curvatures. In the practical operation, an optical fiber will be turned on and transmit the light to the corresponding reference mirror 521, then the processing unit will determine, according to the interference result of the reflected second light and the first light, whether a different optical fiber needs to be turned on (to change the transmission path) in response to a different reflector, for enhancing the measurement accuracy.

Since the relations of other components are similar to the above embodiment, the related descriptions are omitted here for conciseness.

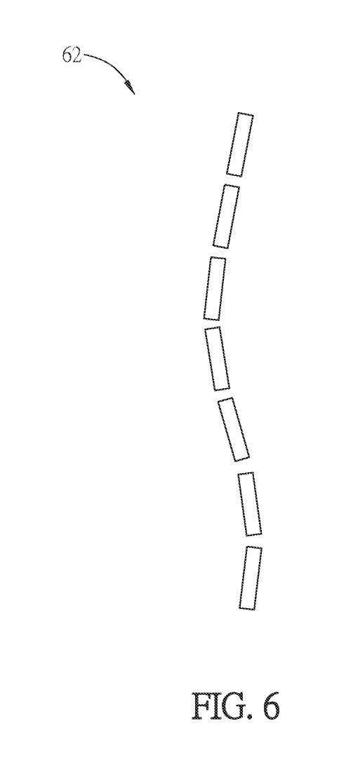

Refer to FIG. 6, which is a schematic diagram of the reference mirror module of the optical measuring device of the fourth embodiment of the invention.

The main difference from the above embodiment is that the reference mirror module 62 of this embodiment is composed of a plurality of digitally-controlled micro-lenses. The advantage of using the digitally-controlled micro-lenses is that each of the lenses can be independently adjusted (in the horizontal position or angle for example), so that the finer adjustment can be made to fit the measurement of irregular surface.

If the measurement is applied to the irregular curved surface, the surface of the under-test object can be divided into a plurality of sub-regions, and the adjustment and measuring can be implemented to each of the sub-regions for at least one time to acquire a better curvature.

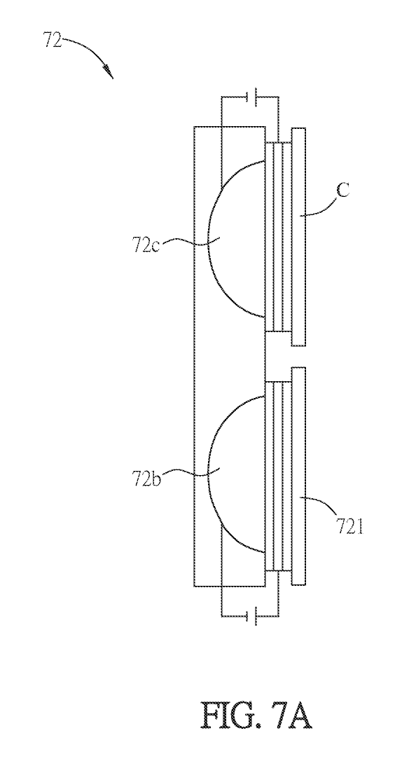

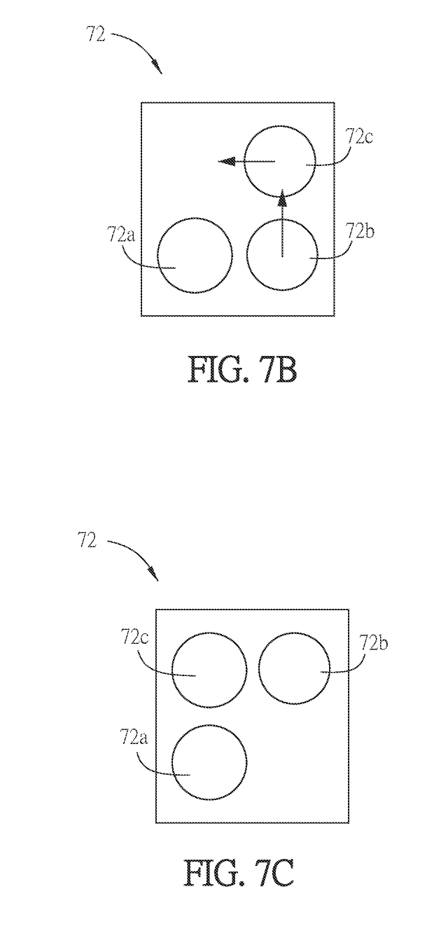

Refer to FIGS. 7A to 7C, which are schematic diagrams of the reference mirror module of the optical measuring device of the fifth embodiment of the invention.

The main difference from the above embodiment is that the reference mirror module 72 is composed of electro-wetting curvature lens and a reflector 721. Besides, the electrode plate C where the electro-wetting curvature lens is disposed can be given a mirror surface treatment so as to achieve the effect similar to the reflector, or the selection of different kinds of liquid can be made to achieve the reflection function. The electro-wetting curvature lens uses the liquid as the zoom lens and is advantageous to the high performance, low cost, small size and less power consumption. The principle thereof is to use the aqueous solution with electroconductivity and the non-conducting oil and vary the contact area of the aqueous solution and the oil by the passing current. Therefore, the increment of the contact area will increase the curvature, so that the focus moves just like the focusing action and the variation of the focal power will be generated.

In detail, the electro-wetting curvature lens can include two conductive layers, and an insulating layer separates the two conductive layers. The conductive layer is made by transparent conductive material such as ITO. The accommodating space formed by the insulating layer and the conductive layers is filled with liquid. The liquid can be mercury, which can form a metal reflective surface. Besides, selecting different liquid will result in different effect. The conductive layer is supplied with the voltage, so that the tortuosity of the liquid is varied due to the difference between the conductivity and insulativity and the focal distance of the lens is thus changed. In other words, the adjusting signal can change the curvature of the adjustable curvature lens according to whether the voltage is applied or not.

Besides, the reference mirror module 72 of this embodiment can be composed of a plurality of electro-wetting curvature lenses 72a, 72b, 72c, and each of the electro-wetting curvature lenses 72a, 72b, 72c can have the same or different disposition. When the curvature needs to be varied, the curvature of the specific electro-wetting liquid surface can be changed by the adjusting signal controlling the applied voltage.

Furthermore, the electro-wetting curvature lenses 72a, 72b, 72c also can move according to the adjusting signal to receive the light and generate the second light (the moving direction is shown by the arrowhead in FIG. 7B).

In other words, this embodiment can use different electro-wetting curvature lenses to control the curvatures of the reflectors to achieve the reflective characteristic the same as or similar to the under-test object for obtaining the more accurate measurement result. Besides, the adjustable curvature lens also can be a dielectrophoretic curvature lens. The dielectrophoresis is to use the interaction between the electric dipole induced by the applied electric field and the applied electric field to drive the particles, and therefore the particles needn't be charged and the dielectrophoresis is driven by AC voltage.

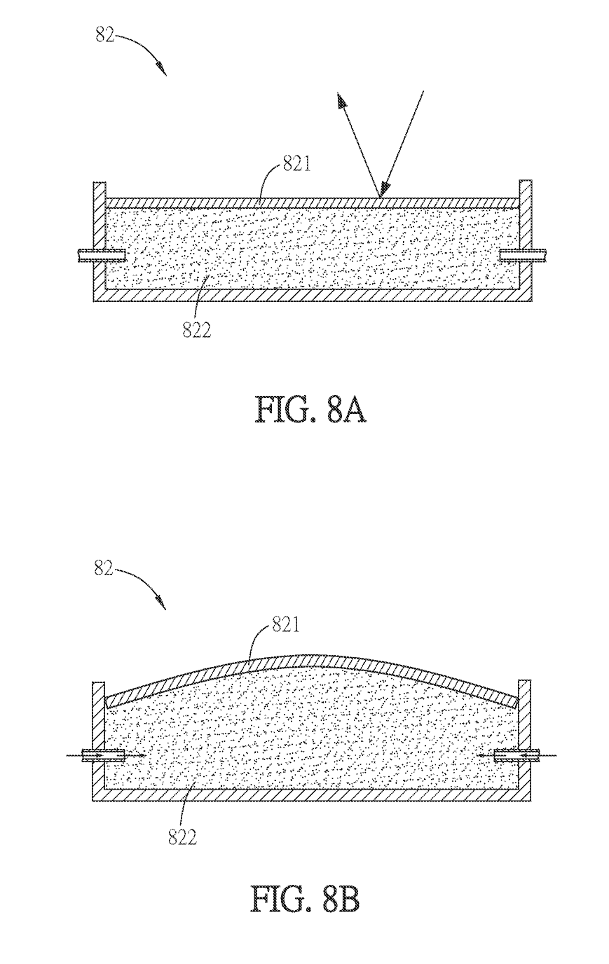

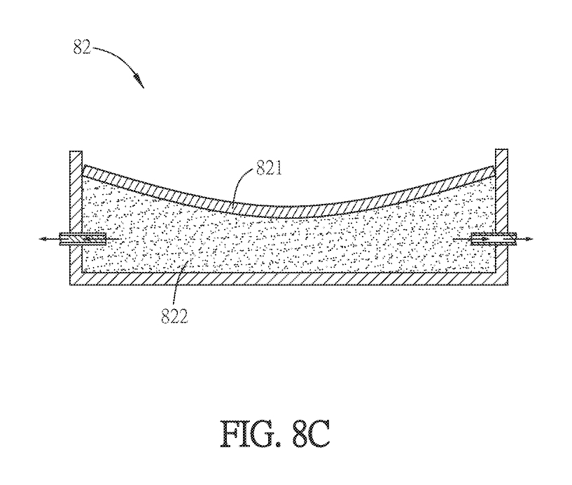

Refer to FIGS. 8A to 8C, which are schematic diagrams of the reference mirror module of the optical measuring device of the sixth embodiment of the invention.

The main difference from the above embodiment is that the reference mirror module 82 of this embodiment is composed a flexible reflector 821 and a fluid chamber 822. The curvature of the reflector 821 of this embodiment can be changed by the amount of the gas or liquid flowing through the flow chamber 822. For example, when more fluid is filled in, the reflector 821 will appear with a convex surface (FIG. 8B). When the fluid is discharged according to the adjusting signal, the reflector 821 will appear with a concave surface (FIG. 8C). Therefore, the processing unit can adjust the curvature of the reference mirror module 82 accordingly.

Since the relations of other components are similar to the above embodiment, the related descriptions are omitted here for conciseness.

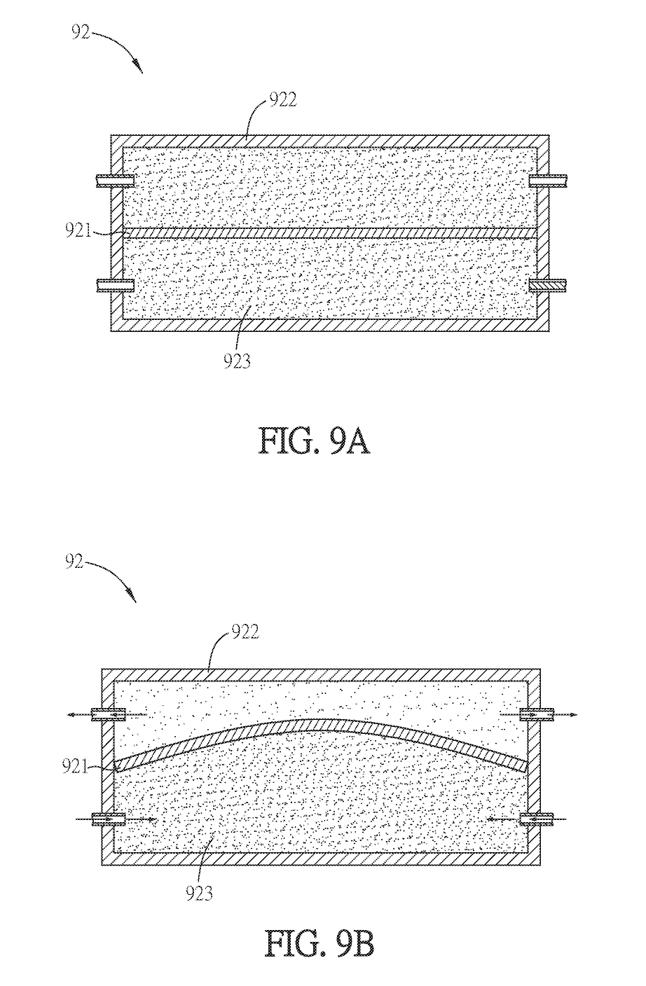

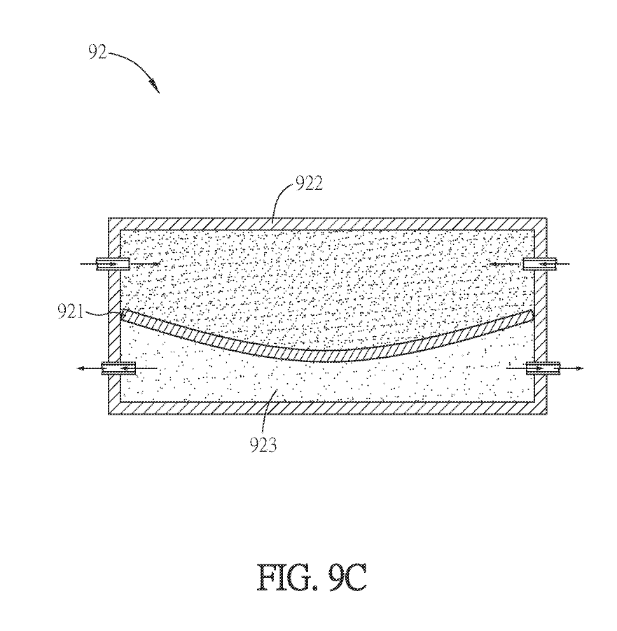

Refer to FIGS. 9A to 9C, which are schematic diagrams of the reference mirror module of the optical measuring device of the seventh embodiment of the invention.

The main difference from the sixth embodiment is that the reference mirror module 92 of this embodiment is composed a flexible reflector 921 and two fluid chambers 922 and 923. Likewise, the curvature of the reflector 921 of this embodiment can be changed by the amount of the gas or liquid flowing through the flow chambers 922 and 923. This kind of configuration of adjusting the curvature of the reflector 921 through two fluid chambers 922 and 923 will achieve wider range of the curvature variation. To be noted, the fluid filled in the upper fluid chamber 922 will affect the reflective path of the second light, so the upper fluid chamber 922 is favorably filled with gas.

Since the relations of other components are similar to the above embodiment, the related descriptions are omitted here for conciseness.

Summarily, in this invention, the optical property of the reference mirror module is adjusted by the interference result between the first and second lights, and therefore the measurement accuracy can be enhanced and the shortcoming that the reference mirror module needs to be, in advance, configured with a proper curvature range in response to different curved surfaces or multi-layer object can be overcome.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. An optical measuring device, comprising:
a light source module providing a light;
a light coupling module;
a reference mirror module; and
a processing unit;
wherein the light of the light source module is transmitted to the reference mirror module and an under-test object through the light coupling module, the light is reflected by the reference mirror module and the under-test object to form a first light beam and a second light, respectively, and the first and second lights are transmitted to the processing unit through the light coupling module;
wherein the processing unit provides an adjusting signal according to the first and second lights, the processing unit transmits the adjusting signal to the reference mirror module, and the reference mirror module adjusts the reference mirror module according to the adjusting signal.

2. The optical measuring device as recited in claim 1, wherein the reference mirror module includes an actuator and a reflector, and the actuator adjusts a curvature of the reflector according to the adjusting signal.

3. The optical measuring device as recited in claim 1, wherein the reference mirror module includes a light path adjusting unit and a plurality of reference mirrors, and the light path adjusting unit matches the second light with one of the reference mirrors according to the adjusting signal.

4. The optical measuring device as recited in claim 3, wherein each of the reference mirrors has different curvature.

5. The optical measuring device as recited in claim 1, wherein the reference mirror module includes an electro-wetting curvature lens or a dielectrophoretic curvature lens.

6. The optical measuring device as recited in claim 1, wherein the under-test object is a spherical body having a plurality of curved surfaces.

7. The optical measuring device as recited in claim 6, wherein the spherical body is an eyeball.

8. An optical measuring method, comprising the steps of:
providing a light to a reference mirror module and another light to an under-test object;
the light reflected by the reference mirror module to form a first light and the other light reflected by the under-test object to form a second light;
the first light and the second light interfering with each other; and
determining whether the interference conforms to a predetermined interference range, if not, generating an adjusting signal according to the interference and adjusting the reference mirror module by the reference mirror module according to the adjusting signal.

9. The optical measuring method as recited in claim 8, wherein the reference mirror module adjusts a curvature of the reference mirror module according to the adjusting signal.

10. The optical measuring method as recited in claim 9, wherein the reference mirror module includes an actuator and a reflector and the steps further comprise:
adjusting a curvature of the reflector by the actuator according to the adjusting signal.

11. The optical measuring method as recited in claim 9, wherein the reference mirror module includes a light path adjusting unit and a plurality of reference mirrors and each of the reference mirrors has different curvature, and the steps further comprise:
matching the second light with one of the reference mirrors by the light path adjusting unit according to the adjusting signal.

12. The optical measuring method as recited in claim 9, wherein the steps further comprise:
forming an image of an under-test surface of the under-test object according to the interference.

13. The optical measuring method as recited in claim 12, wherein the under-test object has a plurality of curved surfaces, and the steps further comprise:
repeating the measuring manner to measure the curved surfaces.

14. The optical measuring method as recited in claim 13, wherein the steps further comprise:
superposing the curved surfaces to form a stereoscopic image.
